(19) United States
(12) Patent Application Publication
(10) Pub. No.: US 2021/0349106 A1
(43) Pub. Date: Nov. 11, 2021

(54) METHOD FOR DIAGNOSING SARS-COV-2 INFECTION

(71) Applicant: University of Science and Technology of China, Anhui (CN)

(72) Inventors: Tengchuan Jin, Anhui (CN); Huan Ma, Anhui (CN); Weihong Zeng, Anhui (CN)

(21) Appl. No.: 17/315,055

(22) Filed: May 7, 2021

(30) Foreign Application Priority Data
May 9, 2020 (CN) 202010390716.2

(51) Int. Cl. G01N 33/68; G01N 33/569

(52) U.S. Cl. CPC G01N 33/6854; G01N 2469/20; G01N 2333/165; G01N 33/56983

(57) ABSTRACT

The present invention discloses a method for diagnosing SARS-CoV-2 infection by detecting SARS-CoV-2 specific IgA in saliva. The diagnosis method can be any method capable of detecting IgA, such as ELISA, co-immunoprecipitation, chemiluminescence and colloidal gold method. The present invention proves that SARS-CoV-2 specific IgA exists in the saliva of COVID-19 patients, which can be used for clinical diagnosis of SARS-CoV-2 infection.

### CROSS-REFERENCE TO RELATED APPLICATIONS

This application claims benefit of Chinese Application No. 202010390716.2 filed on May 9, 2020, which is hereby incorporated herein by reference in their entirety.

### REFERENCE TO SEQUENCE LISTING

The Sequence Listing submitted as a text file named “USTC_100_ST25.txt,” created on Feb. 19, 2021, and having a size of 5,769 bytes is hereby incorporated by reference pursuant to 37 C.F.R. § 1.52(e)(5).

### FIELD OF THE INVENTION

The present invention belongs is in the field of antibody detection, and particularly relates to a method for diagnosing SARS-CoV-2 infection by detecting anti-SARS-CoV-2 IgA in saliva.

### BACKGROUND OF THE INVENTION

At present, for SARS-CoV-2 or 2019-nCoV-induced pneumonia COVID-19, accurate diagnosis and quarantine still are the main methods to control the epidemic. The main method for detecting and diagnosing SARS-CoV-2 infection globally is still to use reverse transcription real-time quantitative PCR (RT-qPCR) to detect viral RNA on oropharyngeal and nasopharyngeal swabs. This method is inconvenient in sampling, not easy to accept, requires the participation of professional medical staff, and has the risk of infection; in addition, since the viral load on the pharynx will decrease significantly as the infection time increases, especially after 15 days of infection, false negative detection results are prone to occur, and the diagnostic accuracy rate is poor.

In addition to the universal RT-qPCR detection methods mentioned above, methods for diagnosing SARS-CoV-2 infection by detecting SARS-CoV-2 specific antibodies, such as immunoglobulin (Ig)M and IgG, in serum are also emerging. However, blood collection is an invasive procedure, and requires the participation of professional medical staff, the method is difficult to apply at places that require fast and large-scale detection, such as airports and customs.

Therefore, a highly accurate detection method for SARS-CoV-2, which is convenient, fast, and involves non-invasive sampling is still needed.

### SUMMARY OF THE INVENTION

The disclosed methods are based at least on the discovery that high content of IgA that specifically binds to SARS-CoV-2 exists in the saliva of patients recovering from COVID-19. Therefore, a convenient, fast and easy-to-accept detection method is provided, whereby SARS-CoV-2 infection is diagnosed by detecting SARS-CoV-2 specific IgA in the saliva. This method is convenient and fast in sampling, does not require the participation of medical staff, and is non-invasive; in addition, in view of the additional discovery that a high level of SARS-CoV-2 specific IgA can be maintained in the infected humans for at least one month, the detection accuracy is high.

There is no report about diagnosing SARS-CoV-2 infection by detecting SARS-CoV-2 specific IgA in saliva.

The present invention is realized through the following technical solutions:

The present invention provides a method for diagnosing SARS-CoV-2 infection, which includes detecting the presence of SARS-CoV-2 specific IgA in saliva of a subject, wherein the presence of the specific IgA indicates SARS-CoV-2 infection. The major structural proteins of SARS-CoV-2 are spike (S), membrane (M), envelope (E), and the nucleocapsid (N) proteins. SARS-CoV-2 genome also encodes open reading frame (ORF), such as ORF 3a, 3b, 0, 7a, 7b, 8, 9b, 9c, 10.

In one embodiment, the detection of SARS-CoV-2 specific IgA in saliva comprises extracting SARS-CoV-2 specific IgA in saliva of a subject with SARS-CoV-2 antigen to form a complex, wherein the SARS-CoV-2 antigen is a viral protein of SARS-CoV-2 that has antigenicity in humans or mammals, such as a spike protein, an N protein, an ORF3b protein, an M protein or an E protein of SARS-CoV-2, or a part of a spike protein, an N protein, an ORF3b protein, an M protein, or an E protein of SARS-CoV-2 that has antigenicity in humans or mammals, such as the receptor binding domain (RBD) of the spike protein of SARS-CoV-2.

In one embodiment, the method further comprises using an anti-IgA antibody (such as an anti-human IgA Fc antibody) with a detectable label (such as a chemiluminescent group or material (such as acridinium ester) or an enzyme) to detect the presence of IgA in the complex.

In one embodiment, the SARS-CoV-2 antigen is coupled to a solid phase carrier (for example, agarose beads, magnetic beads, nitrocellulose membrane, immune plate). The solid phase carrier is used to separate and identify a complex formed by SARS-CoV-2 antigen and SARS-CoV-2 specific IgA from solution.

In one embodiment, detection of SARS-CoV-2 specific IgA in saliva can be accomplished by methods such as chemiluminescence, co-immunoprecipitation, ELISA, colloidal gold etc. These methods are used to detect the presence of SARS-CoV-2 specific IgA in the complex. The sample can be one that is isolated from a subject that may have been exposed to or is suspected of having SARS-CoV-2.

In one embodiment, the subject is a human or mammal

In another aspect, the present invention provides a kit, comprising a solid phase carrier (such as agarose beads, magnetic beads, nitrocellulose membrane, immune plate, etc.) coupled with SARS-CoV-2 antigen and an anti-IgA antibody (for example, an anti-human IgA Fc antibody) with a detectable label, wherein the SARS-CoV-2 antigen is a viral protein of SARS-CoV-2 that has antigenicity in humans or mammals, such as a spike protein, an N protein, an ORF3b protein, an M protein or an E protein of SARS-CoV-2, or a part of a spike protein, an N protein, an ORF3b protein, an M protein, or an E protein of SARS-CoV-2 that have antigenicity in humans or mammals, for example RBD of the spike protein of SARS-CoV-2.

In one embodiment, the kit is used in combination with methods such as chemiluminescence, co-immunoprecipitation, ELISA, or colloidal gold etc.

In one embodiment, the anti-IgA antibody is labeled with a chemiluminescent group or material (for example, acridinium ester) or an enzyme.

In one embodiment, the above-mentioned kit, is used to detect the presence of SARS-CoV-2 specific IgA in the saliva of a subject or to detect SARS-CoV-2 infection in a subject, preferably, the subject is a human or mammal.

The present invention at least has the following technical effects:

1) The present invention uses saliva as the detection object, as opposed to a pharynx swab, the entire sampling process can be completed by the subject to be detected, and the risk of infecting diseases during the sampling process is very low.

2) The present invention uses saliva as the detection object, and the entire sampling process can be completed by the subject to be detected without the participation of professional medical staff. This can save a lot of medical costs.

3) The present invention uses saliva as the detection object. Compared with pharynx swabs and blood, sampling saliva is particularly convenient and fast, and is suitable for places requiring fast detection, such as customs and airports.

4) The present invention uses saliva as the detection object. Compared with pharynx swabs and blood, saliva taking is a non-invasive behavior and is easier to be accepted by the subject to be detected.

5) The present invention which uses SARS-CoV-2 specific IgA detection in saliva, has a higher accuracy rate than a pharynx swab.

All documents mentioned in this specification are incorporated herein in their entirety by reference.

### BRIEF DESCRIPTION OF THE FIGURES

FIG. 1 shows results of IgA in the saliva of healthy humans and COVID-19 recovered patients, detected by co-immunoprecipitation. Lanes 1 and 2 are the saliva detection results for two healthy humans; lanes 3-6 are the saliva detection results for four COVID-19 recovered patients.

FIG. 2 shows the results of IgA in the saliva of healthy humans and COVID-19 recovered patients, detected by chemiluminescence.

FIG. 3 shows diagnosis results of SARS-CoV-2 specific IgA in saliva detected by chemiluminescence method and analyzed by using receiver operating characteristic (ROC) curve.

FIG. 4 shows Mechanism of acridinium ester mediated chemiluminescence, from www.creative-diagnostics.com/Chemiluminescence-Immunoassay-guide.htm.

### DETAILED DESCRIPTION OF THE INVENTION

Methods and kits for detecting the presence of SARS-CO-2 in a sample obtained from a subject, are disclosed. In one embodiment, the subject is a human or mammal. Exemplary animals include domestic animals such as cats and dogs, or animals such a mink, zoo animals such as tigers, lions, gorillas, pumas, cougars, snow leopards, etc. The subject can be symptomatic or asymptomatic. Symptoms of COVID 19 Symptoms include, but are not limited to, fever, congestion in the nasal sinuses and/or lungs, runny or stuffy nose, cough, sneezing, sore throat, body aches, fatigue, shortness of breath, chest tightness, wheezing when exhaling, chills, muscle aches, headache, diarrhea, tiredness, nausea, vomiting, and combinations thereof. The subject is preferably a human.

I. Collection and Treatment of Saliva Samples

Saliva samples for testing are collected from a subject, while ensuring preservation of the quality of the saliva sample by ensuring the sample is collected from a subject who has not engaged in activities such as eating, drinking rinsing the mouth, spitting, etc, at least 10 minutes before the sample is collected. The saliva sample is collected using a suitable sampling container, for example, collection containing having an opening greater than 5 cm and a sealing cap. Saliva is preferably collected by the subject, for instance, by spitting into the saliva collection container. About 0.5, 1, 1.5 or 2 mL of saliva can be collected, and the sealing cap used to close the container. The surface of the container is preferably cleaned after the sealing cap is closed, using a suitable cleaning agent, for example, 75% ethanol. The saliva samples can be at −20° C. or at −80° C. for later detection of SARS-CoV-2. The storage containers are preferably opened in a biological safety cabinet during detection.

II. Detection of SARS-CoV-2 IgA in Saliva Samples

IgA in saliva samples can be detecting using various methods, including, but not limited to co-immunoprecipitation, enzyme-linked immunosorbent assay (ELIZA), colloidal gold and chemiluminescence. The disclosed detection methods preferably use anti-IgA antibody, to detect IgA in the saliva sample. The anti-IgA antibody is selected based on the subject being tested. Thus, where the subject is used, human anti-IgA is selected.

SARS-CoV-2 specific IgA in the saliva sample can be extracted by contacting the saliva sample with a SARS-CoV-2 antigen to form a complex, wherein the SARS-CoV-2 antigen is a viral protein of SARS-CoV-2 that has antigenicity in humans or mammals, for example, a spike protein, an N protein, an ORF3b protein, an M protein or an E protein of SARS-CoV-2, or a part of a spike protein, an N protein, an ORF protein for example, ORF3b protein, an M protein, or an E protein of SARS-CoV-2 that has antigenicity in humans or mammals, such as the receptor binding domain (RBD) of the spike protein of SARS-CoV-2.

A. Coimmunoprecipitation

The SARS-CoV-2 antigen, such as, a spike protein, an N protein, an ORF3b protein, an M protein or an E protein of SARS-CoV-2, or a part of a spike protein, an N protein, an ORF protein for example, ORF3b protein, an M protein, or an E protein of SARS-CoV-2 is coupled to a suitable support such as agarose beads, magnetic beads, nitrocellulose membrane, immune plate, etc.

The antigen-support is brought in contact with the saliva sample, preferably, a diluted saliva sample, for an effective amount of time to ensure binding of the SARS-CoV-2, specific for the antigen selected, as demonstrated in Example 2, below. For example, is the antigen attached to the solid support is RBD, this step ensures the binding of SARS-COV-2 RBD-specific IgA to the RBD on the support. Presence of SARS-COV-2 IgA in the sample can be determined using a suitable assay as SDS-PAGE, the protein in SDS-PAGE is transferred onto a suitable membrane such as a polyvinylidene difluoride (PVDF) membrane or a nitrocellulose, and an anti-IgA antibody is coupled to the SARS-CoV-2 IgA on the membrane by contacting an anti-IgA antibody with the SARS-CoV-2 IgA on the membrane. This is followed by Western blotting. A preferred embodiment of the process steps disclosed herein is demonstrated as a preferred embodiment, in Example 2. PDF and nitrocellulose membranes for Western blotting are commercially available for companies such as Thermofisher Scientific.

B. Chemiluminescence

Chemiluminescence (CL) is defined as the emission of electromagnetic radiation caused by a chemical reaction to produce light. Chemiluminescence immunoassay (CLIA) is an assay that combine chemiluminescence technique with immunochemical reactions. CLIA utilize chemical probes which could generate light emission through chemical reaction to label the antibody.

In this embodiment, the method includes using an anti-IgA antibody, for example anti-human IgA Fc antibody with a detectable label such as a chemiluminescent group, to detect the presence of the SARS-CoV-2 IgA in the saliva sample. CLIA have three different label systems according to the difference of physical chemistry mechanism of the light emission.

The saliva sample is contacted with a solid support which a SARS-CoV-2 antigen, such as, a spike protein, an N protein, an ORF3b protein, an M protein or an E protein of SARS-CoV-2, or a part of a spike protein, an N protein, an ORF protein for example, ORF3b protein, an M protein, or an E protein of SARS-CoV-2 is coupled. After magnetic separation and washing of unbound substances, anti IgA antibody coupled to a marker such as acridinium ester marker is added to the sample, allowing the anti IgA antibody to incubate and bind its antigen present in the sample, and washed again; the substrate solution is added, and then the luminescence reaction of the acridinium ester is detected. This preferred embodiment is exemplified in Example 3. However, it is readily apparent that any known method chemiluminescence methods can be used (Kricka, Analytica chimica acta, 2003, 500(1): 279-286 Vo-Dinh, T. 2003. Chemiluminescence. Encyclopedia of Applied Physics. DOI: 10.1002/3527600434.eap063; Wang, et al., Antibody Detection; Principles and Applications[M]/Advanced Techniques in Diagnostic Microbiology. Springer US, 2013; 53-73 hen W, Jie W U, Chen, et al. Chinese Journal of Analytical Chemistry, 2012, 40(1): 3-10).

(i) Label Chemical Directly Involved in the Light Emission Reaction

This kind of chemical with special structure can transfer to an excited state through chemical reaction. Photons would be released when the chemical fell to ground state from the excited state. The typical chemical is acridinium ester and its derivatives. Exposure of an acridinium ester label to an alkaline hydrogen peroxide solution triggers a flash of light. A subsequent development has been the acridinium sulfonamide ester labels. It is also triggered by alkaline hydrogen peroxide to emit a flash of light. The light emission mechanism of acridinium ester is shown in FIG. 4.

(ii) Enzyme Catalyzed Light Emission Reaction

This type of chemiluminescence utilizes enzymes to label antibody. Technically speaking, it is an enzyme linked immunoassay that uses luminescent chemical as substrate instead of chromogen. The most widely used enzymes are horseradish peroxidase (HRP) and alkaline phosphatase (AP), each has its own luminescent substrates.
(a) Horseradish Peroxidase-Luminol System:

Luminol is a chemiluminescent substrate of HRP. In the presence of peroxide, HRP oxidizes luminol to an excited product called 3-aminophthalate that emits light at 425 nm. The emission continues till 3-aminophthalate decays and enters the ground state. The emitted light may be captured by CCD camera or by exposure to X-Ray film.

(b) Alkaline Phosphatase (AP)-CDP-Star® System.

CDP-Star® (Disodium 2-chloro-5-(4-methoxyspiro{1,2-dioxetane-3,2′-(5′-chloro)tricyclo[3.3.1.1^{3.7}]decan}-4-yl)-1-phenyl phosphate) is a chemiluminescent substrate of AP. CDP-Star® is dephosphorylated by AP to yield meta-stable dioxetane phenolate anion that emits light at 466 nm. The emitted light is stable up to 24 hours allowing for multiple exposures to X-Ray films.

(iii) Redox Reaction Mediated Light Emission Reaction

Another CL system is noteworthy because the reagent is regenerated and thus can be recycled. This system utilizes ruthenium tris-bipyridine (bpy) as label, involves reaction of Ru(bpy)₃³⁺ and Ru(bpy)₃⁺ to produce an excited state of Ru(bpy)₃²⁺, a stable species which decays to the ground state by emitting an 620 nm orange emission. Ru(bpy)₃³⁺ and Ru(bpy)₃⁺ can be electrogenerated from Ru(bpy)₃²⁺ by reduction at approximately −1.3 V and oxidation at approximately +1.3 V

C. ELISA

ELISA (enzyme-linked immunosorbent assay) is a plate-based assay technique designed for detecting and quantifying soluble substances such as peptides, proteins, antibodies, and hormones. In an ELISA, the antigen (target macromolecule) is immobilized on a solid surface (microplate) and then complexed with an antibody that is linked to a reporter enzyme. Detection is accomplished by measuring the activity of the reporter enzyme via incubation with the appropriate substrate to produce a measurable product. There are several formats used for ELISAs. These fall into either direct, indirect, or sandwich capture and detection methods. The key step is immobilization of the antigen of interest, accomplished by either direct adsorption to the assay plate or indirectly via a capture antibody that has been attached to the plate. The antigen is then detected either directly (labeled primary antibody) or indirectly (such as labeled secondary antibody). The most widely used ELISA assay format is the sandwich ELISA assay, which indirectly immobilizes and indirectly detects the presence of the target antigen. This type of capture assay is called a “sandwich” assay because the analyte to be measured is bound between two primary antibodies, each detecting a different epitope of the antigen—the capture antibody and the detection antibody. The sandwich ELISA format is highly used because of its sensitivity and specificity.

An exemplary ELISA assays uses ELISA microtiter plates coated with a SARS-CoV-2 antigen, such as, a spike protein, an N protein, an ORF3b protein, an M protein or an E protein of SARS-CoV-2, or a part of a spike protein, an N protein, an ORF protein for example, ORF3b protein, an M protein, or an E protein of SARS-CoV-2, can be used. A saliva sample collected from a subject is processed and brought in contact with the microtitre plate. After washing the wells to remove all unbound sample material a horseradish peroxidase (HRP) labelled conjugate such as an anti-IgA antibody is added. This conjugate binds to the captured IgA antibodies which bind to the SARS-CoV-2 antigen on the microtitre plate. In a second washing step unbound conjugate is removed. The immune complex formed by the bound conjugate is visualized by adding Tetramethylbenzidine (TMB) substrate which gives a blue reaction product. The conjugate is preferably anti-IgA antibody, for example anti-human IgA Fc antibody. The intensity of this product is proportional to the amount of specific antibodies in the sample. Sulfuric acid is added to stop the reaction. This produces a yellow endpoint color. Absorbance at 450/620 nm is read using an ELBA Microtitre plate reader.

### EXAMPLES

In order to make the objectives, technical solutions and advantages of the present invention clearer, the present invention will be further described in detail below in conjunction with specific examples and with reference to the accompanying drawings.

All healthy humans or COVID-19 recovered patients involved in the present invention signed an informed consent form.

### Example 1: Collection and Treatment of Saliva Samples

1) Two healthy humans and four COVID-19 recovered patients were randomly selected. The subjects are identified as subject No. 1, 2, 3, 4, 5, and 6 to be detected in this experiment. The subjects to be detected were informed that they should not eat, drink or rinse their mouth etc. (activities that will affect the quality of saliva) for at least 10 minutes before saliva sample collection. At the same time, saliva collection containers were distributed to the subject to be detected. The opening of the collection container was preferably greater than 5 cm and had a sealing cap.

2) Saliva was collected by the subject to be detected; about 1 mL of saliva was sufficient. The surface of the container was cleaned after the sealing cap was closed.

3) Saliva samples of the subjects to be detected were collected, and the outer surfaces of the containers were disinfected using 75% ethanol.

4) Saliva samples were stored at −20° C. and opened in a biological safety cabinet during detection.

### Example 2. SARS-CoV-2 Specific IgA in Saliva Samples was Detected by Using Co-Immunoprecipitation Method

1) The receptor binding domain (RBD) protein of the SARS-CoV-2 spike protein (Sequence ID: MT322424.1, see the last page of the specification for the nucleic acid sequence SEQ ID NO. 1) was expressed and purified, and coupled to CNBr-activated Sepharose™ 4B agarose beads (purchased from GE).

2) 1 mL of saliva was taken from each of the above two healthy humans (control) and four COVID-19 recovered patients (that is, subject No. 1, 2, 3, 4, 5, 6) respectively, 4 mL of PBS was added to dilute the saliva and the diluted saliva was transferred to a 10 mL centrifuge tube, 100 μl of the RBD-coupled agarose beads prepared in step 1 of this example was added, mixed well by turning up and down, and incubated at room temperature for 30 min.

3) Each tube was centrifuged at 1000 g for 1 min and the supernatant was discarded. Then 4 mL of PBS was added, mixed by turning up and down 20 times, to wash the beads.

4) Each tube was centrifuged at 1000 g for 1 min, and this Step 3) of Example 2 was repeated 4 times.

5) 300 μL of 0.1 M acetic acid was added into each tube and vortexed for 10 s to elute bound protein.

6) Each tube was centrifuged at 1000 g for 1 min, the supernatant was transferred to a new centrifuge tube, and 60 μl of 1 M Tris-HCl (pH 8.0) was added to neutralize acidity.

7) 20 μL was taken to prepare SDS-PAGE electrophoresis sample, following which, reducing SDS-PAGE electrophoresis was performed. This was followed by Western blotting identification, completed by using the following steps.

8) Transfer to membrane: the protein in SDS-PAGE was transferred to PVDF membrane through wet transferring.

9) The membrane was transferred into a PBS solution containing 5% (weight/volume) milk, and blocked for 1 h at room temperature. Then HRP-coupled anti-human IgA Fc antibody (Boster biological technology) was added and incubated at room temperature for 1 h.

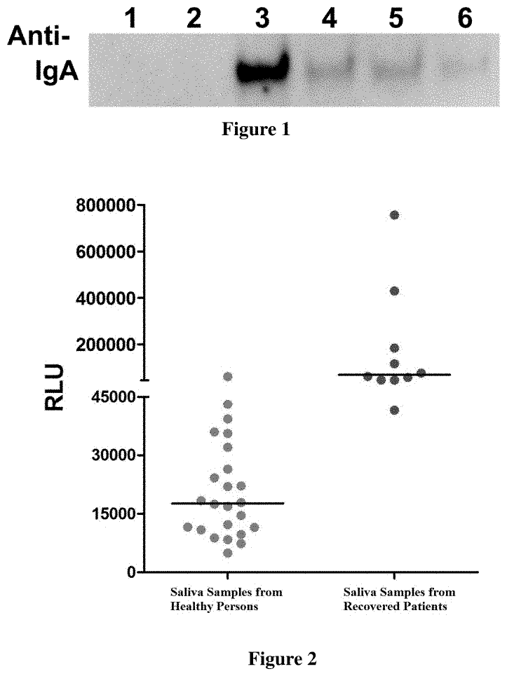

10) The membrane was washed for 5 times with PBS containing 0.1% Tween-20. The membrane was transferred to the photographic plate of the bio-rad gel imaging system, and then a certain amount of substrate was added to develop color (abpbiotech) on the surface of the cover membrane, and photos were taken in the bio-rad gel imaging system. The result is shown in FIG. 1. It can be seen that, bands of heavy chain of SARS-CoV-2 RBD-specific IgA were successfully detected in the saliva of the four recovered patients, whereas, IgA bands were not detected in two healthy human saliva samples used as control.

The above results show the presence of SARS-CoV-2 specific IgA in the saliva of COVID-19 recovered patients. Therefore, the presence of SARS-CoV-2 specific IgA in the saliva can also be detected by methods such as ELISA, colloidal gold and chemiluminescence etc.

### Example 3. Detection of SARS-CoV-2 Specific IgA in Saliva Samples by Chemiluminescence Method

The principle of detection by chemiluminescence method was as follows: the saliva sample to be detected was co-incubated with magnetic beads coated with SARS-CoV-2 RBD, and after magnetic separation and washing of unbound substances, anti-human IgA antibody acridinium ester marker was added to incubate together, and washed again; the substrate solution was added, and then the luminescence reaction of the acridinium ester was detected. If SARS-CoV-2 IgA antibody was present in the sample, magnetic bead coating-SARS-CoV-2 IgA antibody-acridinium ester marker complex can be formed if the SARS-CoV-2 IgA antibody exists in the sample. The luminescence intensity of the acridinium ester is positively correlated with the content of the SARS-CoV-2 IgA antibody, the detection result was indicated by the critical value index (COI).

Kaeser 1000, a fully automatic detection machine, was used in the chemiluminescence detection in this example, and the luminescence value of the negative and positive controls were used for calibration during screening.

The specific steps were:

1) The SARS-CoV-2 RBD purified in Example 2 and magnetic beads (purchased from Wuxi Biomag Biotechnology Co., Ltd., 2.8 micron carboxyl modified magnetic beads, Item number: BMS2800-2A-2 ml) were used to form antigen coated magnetic beads EDC one-step method and EDC/SNHS two-steps method according to the magnetic beads instructions.

2) Saliva samples of 24 healthy humans and 10 COVID-19 recovered patients were collected randomly according to the sampling method of Example 1, and diluted to 40× with PBS respectively.

3) Before being uploaded on the machine, the coated magnetic beads needs to be gently turned up and down about 30 times to evenly disperse the magnetic bead particles. Mixing was not needed to be continued after the coated magnetic beads were loaded for the first time.

4) The reagent position was selected on the instrument operation interface of the automatic detection machine Kaeser 1000, the QR codes on the reagent shelf were scanned, and the reagent shelf was added into the reagent compartment.

5) Sample diluent was prepared according to the sample diluent instruction of the automatic detection machine Kaeser 1000.

6) Cleaning solution was prepared according to the cleaning solution instructions.

7) Substrate solution A and substrate solution B were prepared according to the substrate solution instructions of the automatic detection machine Kaeser 1000.

8) Negative control (SARS-CoV-2 IgA negative serum) and positive control (purified humanized SARS-CoV-2 IgM antibody) were added on the sample shelf and pushed into the sample compartment. Sample types were set as negative control and positive control respectively on the “sample application” interface. The running program was set as follows, and named as SARS-CoV-2 IgA project, SARS-CoV-2 IgA project was selected, negative control and positive control should be conducted for 2 replicates, “Run” was clicked after confirmation.

The above mentioned running program was:

The total incubation time was 15 minutes.

The substances to be detected (negative and positive controls in this step) were transported to the liquid suction point.

The reaction cups were loaded into the running channel.

30 μL of the substance to be detected was drawn into the reaction cup.

The reaction cup was transported to the reagent compartment and 50 μL of reagent R1 was added.

After shaking and mixing, the reaction cup was transported to the incubation compartment and incubated at 37° C. for 10 minutes.

The reaction cup was transported to the washing channel for magnetic separation, the reaction mixture was washed with the washing liquid, and the magnetic separation-washing was repeated 3 times.

The reaction cup was transported to the reagent compartment again, and 50 μL of reagent R2 was added.

After shaking and mixing, the reaction cup was transported to the incubation compartment and incubated at 37° C. for 5 minutes. The reaction cup was transported to the washing channel for magnetic separation, the reaction mixture was washed with the washing liquid, and the magnetic separation-washing was repeated 3 times.

The reaction cup was transported to the substrate channel, 100 μL of substrate solution A was added, shaken and mixed.

The reaction cup was transported to the detection channel, the reaction cup was grabbed to the detection compartment, 100 μL of substrate solution B was added, the luminescence signal was detected immediately, and the COI value of IgA was calculated.

The reaction cup was grabbed to the waste bin.

9) Detection: The saliva sample was placed on the sample shelf (the sample size was greater than 300 μL), pushed into the sample shelf, the sample information was edited on the operation interface, the SARS-CoV-2 IgA project was selected, “Run” was clicked after confirmation. The substance to be detected in this step was saliva sample, and the detection process can be completed within about 20 minutes.

10) Results judgment

When COI<0.8, the SARS-CoV-2 IgA in the sample had no reactivity;

when 0.8 [symbol] COI<1.0, the SARS-CoV-2 IgA in the sample was uncertain;

when COI [symbol] 1.0, the SARS-CoV-2 IgA in the sample was reactive.

The results were shown in FIG. 2. The SARS-CoV-2 specific IgA signal in most COVID-19 recovered patients was significantly higher than those of healthy humans.

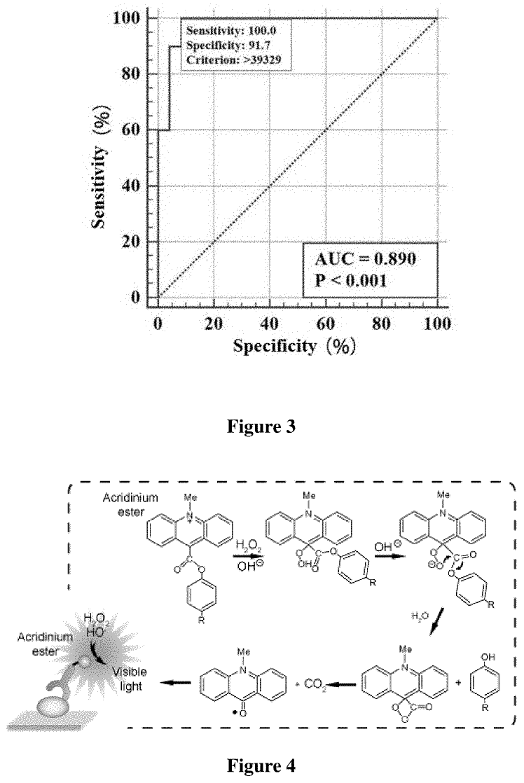

Then MedCalc software was used to perform receiver operating characteristic (ROC) curve analysis based on the detection results. The results were shown in FIG. 3. Through the analysis of the saliva detection results of 10 COVID-19 patients and 24 healthy humans, it was shown that the sensitivity of detection of SARS-CoV-2 infection by SARS-CoV-2 specific IgA was 100%, and the specificity thereof was 91.7%.

Since the testees are COVID-19 recovered patients, the levels of SARS-CoV-2 specific IgA in their bodies at this time are far lower than the levels during the course of the disease. The accuracy will further be improved if COVID-19 infected patients were detected.

The specific examples described above further describe the objective, technical solutions and beneficial effects of the present invention in detail. It should be understood that the above mentioned are only specific examples of the present invention and are not intended to limit the present invention. Any modification, equivalent replacement, improvement, etc. made within the spirit and principle of the present invention, shall be included in the protection scope of the present invention.

Nucleic acid sequence of RBD
(SEQ ID NO: 1)
CAACCAACAGAATCTATTGTTAGATTTCCTAATATTACAAACTTGTGCCCT
TTTGGTGAAGTTTTTAACGCCACCAGATTTGCATCTGTTTATGCTTGGAAC
AGGAAGAGAATCAGCAACTGTGTTGCTGATTATTCTGTCCTATATAATTCC
GCATCATTTTCCACTTTTAAGTGTTATGGAGTGTCTCCTACTAAATTAAAT
GATCTCTGCTTTACTAATGTCTATGCAGATTCATTTGTAATTAGAGGTGAT
GAAGTCAGACAAATCGCTCCAGGGCAAACTGGAAAGATTGCTGATTATAAT
TATAAATTACCAGATGATTTTACAGGCTGCGTTATAGCTTGGAATTCTAAC
AATCTTGATTCTAAGGTTGGTGGTAATTATAATTACCTGTATAGATTGTTT
AGGAAGTCTAATCTCAAACCTTTTGAGAGAGATATTTCAACTGAAATCTAT
CAGGCCGGTAGCACACCTTGTAATGGTGTTGAAGGTTTTAATTGTTACTTT
CCTTTACAATCATATGGTTTCCAACCCACTAATGGTGTTGGTTACCAACCA
TACAGAGTAGTAGTACTTTCTTTTGAACTTCTACATGCACCAGCAACTGTT
TGTGGACCTAAAAAGTCTACTAATTTGGTTAAAAACAAATGTGTCAATTTC
AACTTCAATGGTTTAACAGGCACAGGTGTTCTTACTGAGTCTAACAAAAAG
TTTCTGCCTTTCCAACAATTTGGCAGAGACATTGCTGACACTACTGATGCT
GTCCGTGATCCACAGACACTTGAGATTCTTGACATTACACCATGTTCT

We claim:

1. A method for diagnosing SARS-CoV-2 infection, comprising detecting the presence of SARS-CoV-2 specific IgA in saliva of a subject, wherein the presence of the specific IgA indicates SARS-CoV-2 infection.

2. The method of claim 1, wherein detection of SARS-CoV-2 specific IgA in saliva comprises extracting SARS-CoV-2 specific IgA from the saliva of the subject with a SARS-CoV-2 antigen to form a complex, wherein the SARS-CoV-2 antigen is a viral protein of SARS-CoV-2 that has antigenicity in humans or mammals, such as a spike protein, an N protein, an ORF3b protein, an M protein or an E protein of SARS-CoV-2, or a part of a spike protein, an N protein, an ORF3b protein, an M protein or an E protein of SARS-CoV-2 that has antigenicity in humans or mammals, such as RBD of the spike protein of SARS-CoV-2.

3. The method of claim 2, further comprising using an SARS-CoV-2 antigen with a detectable label to detect the presence of IgA in the complex.

4. The method of claim 2, wherein the SARS-CoV-2 antigen is coupled to a solid phase carrier selected from the group consisting of agarose beads, magnetic beads, nitrocellulose membranes, and immune plates.

5. The method of claim 1, wherein detection of SARS-CoV-2 specific IgA in saliva is achieved by a chemiluminescence, co-immunoprecipitation, ELISA or colloidal gold method.

6. The method of claim 1, wherein the subject is a human or a mammal.

7. A kit comprising a solid-phase carrier coupled with a SARS-CoV-2 antigen and an anti-IgA antibody with a detectable label wherein the SARS-CoV-2 antigen is a viral protein of SARS-CoV-2 that has antigenicity in humans or mammals, such as a spike protein, an N protein, an ORF3b protein, an M protein or an E protein of SARS-CoV-2, or a part of a spike protein, an N protein, an ORF3b protein, an M protein or an E protein of SARS-CoV-2 that has antigenicity in humans or mammals, such as RBD of the spike protein of SARS-CoV-2.

8. The kit of claim 7 which is used in combination with a chemiluminescence, co-immunoprecipitation, ELISA or colloidal gold method.

9. The kit of claim 7, wherein the anti-IgA antibody is labeled with a chemiluminescent group, acridinium ester or an enzyme.

10. The kit of claim 7, which is used to detect presence of SARS-CoV-2 specific IgA in saliva of a subject, preferably, the subject is a human or mammal.

11. The method of claim 3, wherein the anti-IgA antibody is anti-human IgA Fc antibody.

12. The method of claim 3, wherein the detectable label is chemiluminescent group, acridinium ester or an enzyme.

13. The kit of claim 7 wherein the solid phase carrier is selected from the group consisting of agarose beads, magnetic beads, nitrocellulose membranes and immune plates.

14. The kit of claim 7, wherein the anti-IgA antibody is anti-human IgA Fc antibody.


SEQUENCE LISTING (1 sequence)

SEQ ID NO: 1 - DNA, 813 bp, 2019-nCov
caaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttct